(19) United States
(12) Patent Application Publication
(10) Pub. No.: US 2021/0289815 A1
(43) Pub. Date: Sep. 23, 2021

(54) FAST RELEASE BENZOIC ACID IN FEED

(71) Applicant: DSM IP ASSETS B.V., Heerlen (NL)

(72) Inventors: Laure CLASADONTE, Kaiseraugst (CH); Elger FUNDA, Kaiseraugst (CH); Nicholas HAGEMANN, Kaiseraugst (CH); Carsten PETERSEN-PAUSA, Kaiseraugst (CH); Rainer STEBNER, Kaiseraugst (CH)

(21) Appl. No.: 17/255,668

(22) PCT Filed: Jun. 28, 2019
(86) PCT No.: PCT/EP2019/067320

§ 371 Date: Dec. 23, 2020

(30) Foreign Application Priority Data
Jun. 29, 2018 (EP) 18180693.6
Apr. 16, 2019 (EP) 19169404.1

(51) Int. Cl. A23K 20/111; A23K 40/10; A23K 20/158; A23K 50/30; A61K 9/00; A61K 9/16; A61K 31/192

(52) U.S. Cl. CPC A23K 20/111; A23K 40/10; A23K 20/158; A61K 9/1617; A61K 9/0056; A61K 9/1688; A61K 31/192; A23K 50/30

(57) ABSTRACT

The present invention relates to the use of benzoic acid prills in the feed industry. Despite of having a reduced surface area/volume ratio, the benzoic acid prills as used in the context of the present invention release benzoic acid faster than benzoic acid flakes. Compositions comprising such prills are useful for balancing the microflora in an animal's gastro-intestinal system, for acidification of feed and/or urine, and/or for reducing digestive disorders of an animal.

### TECHNICAL FIELD

The present invention relates to the use of benzoic acid in the feed industry.

### BACKGROUND OF THE INVENTION

Benzoic acid is currently being used in the feed industry. It is a colorless solid melting at about 122° C.

Crystalline benzoic acid is commercially available as white scales/flakes or as needle crystals. Commercially available feed premixes contain benzoic acid flakes. Needle crystals are not suitable for feeding animals.

WO 2012/049013 A1 discloses the use of benzoic acid in a feed concentrate for the prevention and/or treatment of urolithiasis, for reducing the number and/or size of urine uroliths, and/or for improving ruminant welfare.

WO 98/08499 A1 relates to a composition comprising benzoic acid for use as a medicament for animals, wherein said acid functions as an antibiotic, promotes growth, decreases feed conversion ratio and improves digestibility of amino acids administered in animal feeds. The benzoic acid can be administered to the animal either in its feed composition or in its drinking water.

Benzoic acid is poorly soluble in water. There is a need for controlling the release of benzoic acid such that benzoic acid is released faster. Thereby, the envisaged technical solution must be sustainable, cost effective and must fulfil regulatory requirements which apply for the feed industry. Envisaged is a composition which is non-toxic, storage stable, safe to handle (e.g. reduced odour) and/or which shows reduced compaction when exposed to pressure.

### SUMMARY OF THE INVENTION

Ammonia emission is a major issue. Technical solutions are needed to make livestock farming more sustainable.

Ammonia emission from slurry is affected by urinary pH. The lower the urinary pH, the lower the ammonia emission from slurry. Galassi et al. have investigated the “Effects of benzoic acid on nitrogen, phosphorus and energy balance and on ammonia emission from slurries in the heavy pig” (Gianluca Galassi, Luca Malagutti, Stefania Colombini, Luca Rapetti & G. Matteo Crovetto, Italian Journal of Animal Science, 10:3, DOI: 0.4081/ijas.2011.e38). In their study, Galassi et al. used benzoic acid flakes as commercially available under the tradename VevoVitall® (DSM Nutritional products, Switzerland).

Adding benzoic acid to the diet of growing pigs markedly acidifies the pig's urine, but only slightly reduces blood pH. Interestingly, the lower urine pH is attributable to the fact that the ingested benzoic acid is absorbed in the first tract of the small intestine. This differentiates benzoic acid over other organic acids (cf. literature cited in Galassi et al.).

To achieve absorption in the first tract of the small intestine, benzoic acid needs to be released (e.g. dissolved) relatively fast. Unsolved, solid benzoic acid passes the small intestine without being absorbed (i.e. is not bioavailable). Thus, there is a need for controlling the release (e.g. the dissolution) of benzoic acid such that benzoic acid is released faster.

The problems underlying the present invention are solved by using benzoic acid prills instead of benzoic acid flakes.

Benzoic acid prills as such are known. Example 1 of WO 2002/051785 discloses benzoic acid prills that were prepared by means of jet-prilling technology. WO 2002/051785 does not disclose the use of benzoic acid prills in the feed industry.

Benzoic acid prills have a smaller surface area/volume ratio than benzoic acid flakes. It is therefore to be expected that benzoic acid prills dissolve more slowly than benzoic flakes. Surprisingly, the opposite is the case: benzoic acid prills release benzoic acid faster than benzoic acid flakes.

In addition, benzoic acid prills smell less than benzoic acid flakes, and safety at work is improved because the amount of dust is reduced in comparison with commercially available benzoic acid flakes. Furthermore, compaction upon exposure to pressure (e.g. in a silo or when piling bags) is reduced in comparison with benzoic acid flakes. Finally, benzoic acid prills have excellent flowability.

Adding benzoic acid prills to a feed premix or to compound feed has many advantages: it is an easy and cheap solution, the obtained mixture is resistant to de-mixing, and there are no regulatory concerns about the toxicity of auxiliary compounds because there are no auxiliary compounds. Finally, it is a sustainable solution as it allows for a reduction of ammonia emission from slurry.

The preferred average particle size D (v,0.5) of the benzoic acid prills depends on the animal to be fed: premixes for larger animals (such as pigs) may contain larger particles than premixes for smaller animals (such as chicken). In case of premixes for chicken, the benzoic acid prills have an average particle size D (v,0.5) of not more than 2 mm, measured by Laser Diffraction; Malvern Mastersizer 2000, MIE volume distribution.

### DETAILED DESCRIPTION OF THE INVENTION

The benzoic acid prills of the invention are obtainable by prilling. “frilling” is a process wherein a molten solid is converted into particles.

The benzoic acid prills of the invention are spherical. In the context of the present invention, “spherical” means that the benzoic acid prills of the invention roll down a plane that is covered with filter paper and that is inclined at an angle of 15.5 degrees. Preferably, the plane is covered with filter paper having a pore size of at least 10 μm, preferably of 15-19 μm such as a Schleicher and Schuell 520 BII filter paper.

According to a preferred embodiment of the invention, a prill is designated as spherical if the length-width ratio (i.e. the ratio of the length (largest dimension) of the particle, divided by the width (smallest dimension, determined at an angle of 90° to the length)) is less than about 1.4. Preferably the length-width ratio of a spherical particle is less than about 1.3, more preferably less than about 1.2, even more preferably less than about 1.1 and in particular less than about 1.05.

According to the most preferred embodiment of the invention, a prill is designated as spherical if it forms an ellipsoid of revolution (spheroid), wherein the length-width ratio (i.e. the ratio of the length (largest axis) of the prill, divided by the width (smallest axis, determined at an angle of 90° to the length)) is less than about 1.4. Preferably the length-width ratio of a spherical particle is less than about 1.3, more preferably less than about 1.2, even more preferably less than about 1.1 and in particular less than about 1.05. The benzoic acid prills of the invention have typically neither edges nor spikes.

The person skilled in the art is familiar with prilling technology. It is applied in various industries and is used, for example, in the production of fertilizers such as urea. The use of prilling for the manufacture of a composition which releases benzoic acid faster is novel and surprising.

Prilling is a process which is based on spray cooling. To obtain benzoic acid prills, liquid (i.e. molten) benzoic acid is sprayed through or into a cooling medium. Benzoic acid is not very oxygen sensitive. Thus, a wide variety of cooling media can be used, including air. Spraying is typically done in a prilling tower. A preferred supplier of prilling towers is Kreber (Vlaardingen, Netherlands).

In the prilling tower, when falling through a suitable cooling medium, liquid (i.e. molten) benzoic acid drops solidify. Surface tension causes the liquid benzoic acid to adopt a more or less spherical shape as this results in the smallest surface area/volume ratio. To obtain more spherical drops, the flow pattern of cooling air inside the prilling tower should be controlled by suitable means. In one embodiment of the invention, the technology as disclosed in DE 1 230 402 B is used.

If processing conditions are not fully optimized, uneven crystal-shaped particles are obtained as a by-product. These uneven crystal-shaped particles are not spherical and are therefore not benzoic acid prills as used in the context of the present invention. Ideally, process conditions are optimized such that this by-product no longer occurs.

Alternatively, after the actual prilling process, uneven crystal-shaped benzoic acid particles are separated from the benzoic acid prills. This can be done manually, if necessary by use of a microscope. Preferably, however, a device for dry separation of spherical particles from particles having non-spherical shape is used. The functionality of such devices is based on the difference between the sliding friction of non-spherical particles and the rolling friction of spherical particles. Due to the difference between the friction coefficients, the two types of particles move along different trajectories, and thus become separable. Such devices are described for example in Zeno Sparchez, “Devices for Dry Separation of the Spherical Particles from those having Non Spherical Shape from a Metallic Powder”, Advanced Material Research, ISSN: 1662-8985. Vol. 23. pp 91-94 (2007).

The present invention also relates to a method of producing benzoic acid prills, said method comprising the steps:
(i) providing particles by spray cooling molten benzoic acid;
(ii) removing non-spherical particles from the particles provided in step (i).

Preferably, step (i) is done by spraying molten benzoic acid into a gaseous cooling medium, wherein said gaseous cooling medium is preferably air. Also preferably, the difference between the sliding friction of non spherical particles and the rolling friction of spherical particles is used in step (ii) for separating non-spherical particles from spherical particles.

Non-spherical particles which have been removed in step (ii) may be discharged. Preferably, however, said non-spherical particles are fed back into the prilling process. In other words, the non-spherical particles which have been removed in step (ii) are molten once again before being sprayed for a second time into or through a gaseous cooling medium such as air. In this preferred embodiment, nothing needs to be discharged.

In one embodiment of the invention, the term “benzoic acid prills” refers to benzoic acid prills which comprise at least 95 weight-% benzoic acid, preferably at least 98 weight-% benzoic acid, more preferably at least 99 weight-% benzoic acid and even more preferably at least 99.5 weight-% benzoic acid and most preferably at least 99.9 weight-% benzoic acid, based on the total weight of the prills. Preferably, benzoic acid prills as used in the present invention are free of fat-soluble auxiliary compounds.

Preferably, the term “benzoic acid prills” refers to spherical particles which have neither edges nor spikes. Thus, in a preferred embodiment of the invention, the term “benzoic acid prills” refers to particles which are spherical, which have neither edges nor spikes and which comprise at least 95 weight-% benzoic acid, preferably at least 98 weight-% benzoic acid, more preferably at least 99 weight-% benzoic acid and even more preferably at least 99.5 weight-% benzoic acid and most preferably at least 99.9 weight-% benzoic acid, based on the total weight of the prills.

In one embodiment of the invention, the term “benzoic acid prills” refers to benzoic acid prills which have average particle size D (v,0.5) from 0.5 mm to 6 mm, more preferably from 0.5 mm to 5 mm and most preferably from 0.5 mm to 2 mm, measured by Laser Diffraction; Malvern Mastersizer 2000, MIE volume distribution. D (v, 0.5) is the size at which 50% of the sample is smaller and 50% is larger. When used for feeding chicken, benzoic acid prills are used which have average particle size D (v,0.5) of 2 mm or less.

In one embodiment of the invention, less than 4% of the benzoic acid prills are in the size category 0.425 mm-0.600 mm. This means that the total volume of all benzoic acid prills with diameters in this range represents less than 4% of the total volume of all particles in the distribution. In another embodiment of the invention, less than 3%, preferably less than 2%, and most preferably less than 1% of benzoic acid prills are in the size category 0.425 mm-0.600 mm. In an also preferred embodiment, the benzoic acid prills as herein described have no particles which have a diameter of less than 0.4 mm (measured by Laser Diffraction; Malvern Mastersizer 2000).

Benzoic acid prills as herein described have excellent flowability. In a preferred embodiment of the invention, 100 g benzoic acid prills being filled into a conical funnel flow through the funnel's orifice within 5 seconds or less, wherein said funnel is made of electropolished metal and wherein the angle between longitudinal axis of funnel and the funnel's wall is 10.5 degrees, and wherein the funnel's orifice has an inside diameter of 11.3 mm.

The present invention relates to a method of releasing benzoic acid faster in the intestine of an animal. Surprisingly, faster release of benzoic acid is achieved when benzoic acid prills instead of benzoic acid flakes are fed to an animal.

In the context of the present invention, “faster release of benzoic acid” means that the rate and/or the extent of in vitro dissolution of benzoic acid in a physiologically-relevant dissolution fluid is higher when compared to commercially available benzoic acid flakes. A benzoic acid flake is a flat and/or thin piece.

The present invention also relates to a method of increasing the dissolution rate of benzoic acid, wherein benzoic acid is provided as benzoic acid prills. A preferred embodiment of the invention relates to a method of increasing the dissolution rate of benzoic acid in gastric fluid, wherein benzoic acid is provided as benzoic acid prills and wherein said gastric fluid is preferably simulated gastric fluid (SGF). An also preferred embodiment of the invention relates to a method of increasing the dissolution rate of benzoic acid, wherein benzoic acid is provided as benzoic acid prills and wherein said dissolution rate is preferably measured at predetermined parameters in simulated gastric fluid (SGF).

Preparation of simulated gastric fluid is described e.g. in US pharmacopeia: Dissolve 2.0 g of sodium chloride and 3.2 g of purified pepsin, that is derived from porcine stomach mucosa, with an activity of 800 to 2500 units per mg of protein, in 7.0 mL of hydrochloric acid and sufficient water to make 1000 mL. This test solution has a pH of about 1.2.

Parameters which are relevant in this kind of dissolution test include temperature, stirring speed and flow rate. When comparing the dissolution rate of two or more samples, these parameters must be kept at predetermined values. In one embodiment, at least 60%, preferably at least 65% of the prills' benzoic acid has been dissolved after 30 minutes in simulated gastric fluid when measuring dissolution in a USP-4 release tester, using the following parameters:
Test temperature: 37.0° C.
Pump delivery: 8 ml/min.
Cell: 12 mm
Buffer: HCl pH 2

One embodiment of the invention relates to benzoic acid prills for use in the enhancement of the bioavailability of orally administered benzoic acid, wherein said benzoic acid is administered to an animal, preferably to a mammal and most preferably to domestic animal which is a mammal. Said embodiment also relates to a method of treating an animal, wherein benzoic acid prills are orally administered to said animal in order to enhance bioavailability of orally administered benzoic acid. It also relates to the use of benzoic acid prills for enhancing the bioavailability of orally administered benzoic acid, wherein said benzoic acid is administered to an animal, preferably to a mammal and most preferably to domestic animal which is a mammal. Another embodiment of the invention relates to the use of benzoic acid prills for enhancing the bioavailability of orally administered benzoic acid by increasing the amount of dissolved benzoic acid in the first tract of the small intestine of an animal (preferably of a mammal) after oral ingestion of a predetermined amount of solid benzoic acid.

Enhanced bioavailability results in a more effective acidification of the pH of an animal's urine—and lowering the urinary pH reduces ammonia emission from slurry.

Therefore, the present invention also relates to the use of benzoic acid prills for reducing ammonia emission from slurry of an animal, wherein benzoic acid prills are fed to said animal. It also relates to the use of benzoic acid prills for reducing ammonia emission from slurry, wherein said slurry originates from an animal, preferably from a mammal and even more preferably from a pig.

The present invention also relates to the use of benzoic acid prills as described in the present patent application for acidification of feed and/or urine. In a preferred embodiment, the invention relates to the use of benzoic acid prills for acidification of urine. In this embodiment, benzoic acid prills are fed to the animal whose urine is to be acidified. In the context of the present invention, the term “animal” refers preferably to a domestic animal, more preferably to a farm animal and even more preferably to a chicken and/or to a pig. Most preferably, the term “animal” refers to a pig.

The present invention also relates to the improvement of an animal's gut health. A preferred embodiment of the invention relates to the use of a composition comprising benzoic acid prills for balancing the microflora in an animal's gastro-intestinal system and/or to reduce digestive disorders of an animal. The invention also relates to a method for improving the gut health of an animal, said method comprising the step of oral administration of benzoic acid prills to an animal.

The present invention also relates to animal food comprising benzoic acid prills. In the context of the present invention, “animal food” refers to a composition which can be fed to an animal such as a pig or a chicken. Animal food may be e.g. a feed additive, a premix or feed. A preferred embodiment of the invention relates to a feed additive or a premix comprising benzoic acid prills, at least one flavouring substance, at least one vitamin and/or at least one mineral salt. Said at least one flavouring substance is preferably an essential oil. Preferred essential oils are thymol and/or eugenol. Thus, a preferred embodiment of the invention relates to a feed additive or a premix comprising benzoic acid prills and at least one essential oil, wherein said at least one essential oil is thymol and/or eugenol.

The present invention also relates to feed comprising benzoic acid prills. Feed as used in the feed industry typically comprises cereals, flour mill by-products, fat and/or proteins. Thus, one embodiment of the invention relates to feed comprising benzoic acid prills and at least one further ingredient, wherein said at least one further ingredient is preferably selected from the group consisting of cereals, flour mill by-products, fat and proteins. One ton feed comprises preferably from 100 mg to 5 kg benzoic acid prills, more preferably from 110 mg to 4 kg benzoic acid prills and most preferably from 120 mg to 3 kg benzoic acid prills. Said amounts may be higher if the feed is meant for pigs: one ton pig feed comprises preferably from 1 kg to 15 kg benzoic acid prills, more preferably from 2 kg to 11 kg benzoic acid prills and most preferably from 3 kg to 10 kg benzoic acid prills.

The present invention also relates to the use of benzoic acid prills as described in the present patent application as flavouring compound and/or as growth supporter or growth promoter.

The present invention also relates to a process for the preparation of animal food, said process comprising the step of adding benzoic acid prills to a composition which can be fed to an animal, said animal being preferably a pig or a chicken. In a preferred embodiment, said process for the preparation of animal food is a process for the preparation of a feed additive, a premix or feed.

The present invention also relates to the following embodiments:
1. Method of controlling the release of benzoic acid in the intestine of an animal, wherein the surface area/volume ratio of crystalline benzoic acid is reduced by providing benzoic acid prills.
2. Composition comprising benzoic acid prills for use in balancing the microflora in an animal's gastro-intestinal system and/or for use in reducing digestive disorders of an animal.
3. Composition according to embodiment 2, for use in balancing the microflora in the gastro-intestinal system of a chicken or a pig and/or for use in reducing digestive disorders of a chicken or a pig.
4. Animal food comprising benzoic acid prills.
5. Animal food according to embodiment 4, wherein said animal food is a feed additive, a premix or feed.
6. Animal food according to embodiment 4 or 5, said animal food further comprising at least one flavouring substance, cereals, flour mill by-products, at least one vitamin, at least one mineral salt and/or proteins.
7. Animal food according to any one of embodiments 4 to 6, said animal food further comprising at least one essential oil, wherein said essential oil is preferably thymol and/or eugenol.
8. Process for the preparation of animal food, said process comprising the step of adding benzoic acid prills to a composition which can be fed to an animal.
9. Process according to embodiment 8, wherein said composition is a feed additive, a premix or feed.
10. Method, composition, animal food or process according to any one of the preceding embodiments, wherein said benzoic acid prills comprise at least 95 weight-% benzoic acid, preferably at least 98 weight-% benzoic acid, more preferably at least 99 weight-% benzoic acid, even more preferably at least 99.5 weight-% benzoic acid and most preferably at least 99.9 weight-% benzoic acid, based on the total weight of the prills.
11. Method, composition, animal food or process according to any one of the preceding embodiments, wherein said benzoic acid prills are free of fat-soluble auxiliary compounds.
12. Method, composition, animal food or process according to any one of the preceding embodiments, wherein said benzoic acid prills are flowable and/or spherical.
13. Method, composition, animal food or process according to any one of the preceding embodiments, wherein said benzoic acid prills have a mean particle diameter, determined by screen analysis, from 0.5 mm to 6 mm, more preferably from 0.5 mm to 5 mm and most preferably from 0.5 mm to 2 mm.
14. Method, composition, animal food or process according to any one of the preceding embodiments, wherein said benzoic acid prills are obtained by spray cooling, and wherein said benzoic acid prills are preferably obtained by spraying liquid benzoic acid into a gaseous cooling medium, said gaseous cooling medium preferably comprising nitrogen and/or oxygen.
15. Use of benzoic acid prills for acidification of feed and/or urine.
16. Use of benzoic acid prills for reduction of ammonia emission from slurry.
17. Use according to embodiment 16, wherein said slurry originates form an animal, preferably from a mammal and even more preferably from a pig.

### FIGURES

FIG. 1 show the result of the compaction test done in example 1. FIG. 1a shows the compaction behavior of a composition which essentially consists of benzoic acid prills. FIG. 1b shows the compaction behavior of commercially available benzoic acid flakes. The comparison of FIG. 1a with FIG. 1b shows one of the benefits of using benzoic acid prills in the feed industry: discharging benzoic acid prills out of bins, silos etc. is easy because compaction during storage is reduced or even absent.

FIG. 2 show particles consisting of benzoic acid. FIG. 2a shows particles produced in a prilling tower. Some of the particles are benzoic acid prills whereas others are a non-spherical by-product. After removal the non-spherical particles, the particles shown in FIG. 2b were obtained. FIG. 2c shows, as a comparison, benzoic acid flakes as commercially available under the tradename VevoVitall®. These flakes are neither spherical nor produced by prilling.

FIG. 3 shows the result of the dissolution tests done in example 3. On the x-axis, the duration of the dissolution test is shown in minutes. On the y-axis, the amount of dissolved benzoic acid is shown in percentages. The dissolution of three kinds of particles is shown in FIG. 3: flakes, prills and non-spherical by-product. After 60 minutes, less than 90% of the benzoic acid flakes (VevoVitall®) had been dissolved. The non-spherical by-product of the prilling process showed a similar release. Surprisingly, the benzoic acid prills of the invention dissolved quicker. After 30 minutes, 66.71% of the benzoic acid prills were dissolved whereas only 57.6% of the benzoic acid flakes were dissolved after 30 minutes.

FIG. 4 shows the result of the test done in example 4. The first line of pictures in FIG. 4 is the backside view of a jolting volumeter after 0 taps, after 375 taps, after 1125 taps and after 1500 taps. The second line of pictures in FIG. 4 shows the upper part of the same jolting volumeter after the same number of taps whereas the third line of pictures in FIG. 4 shows the lower part of the same jolting volumeter after the same number of taps.

FIG. 4 illustrates that the benzoic acid prills of the invention are suitable for producing animal food such as a feed additive.

### EXAMPLES

### Example 1

Particles consisting of benzoic acid were produced in a prilling tower by spraying liquid benzoic acid into a gaseous cooling medium (air). Physical characteristics of the obtained particles were measured and compared with the physical characteristics of commercially available benzoic acid flakes (VevoVitall®). The result of this comparison is summarized in below TABLE 1.

TABLE 1

| | | | Benzoic Acid particles |
|---|---|---|---|
| | | Benzoic Acid | produced in prilling |
| Sample | | Flakes VevoVitall ® | tower |
| composition | | 100% benzoic acid | 100% benzoic acid |
| Aspect | | white coarse powder/ | Mixture of white |
| | | small broken flakes | spherical particles and |
| | | | non-spherical crystals |
| Smell | | very strong | typical but less strong |
| Flowability through Agway | | 6/high | 5/high |
| funnel, orifice 11.3 mm | | | |
| (sec/100 g) | | | |
| Dusting Heubach (mg/25 | | 28/low | 3/very low |
| g)/Rating | | | |
| Bulk density (kg/l) | | 0.60 | 0.65 |
| Tapped density (kg/l) | | 0.68 | 0.68 |
| Particle size | | | |
| Average D (v, 0.5) | | 1.3 mm | 1.12 mm |
| <0.425 | mm | 1.7% | 0% |
| 0.425-0.600 | mm | 4.3% | 0.2% |
| 0.600-0.850 | mm | 10.9% | 12.3% |
| >0.850 | mm | 83.1% | 87.5% |

| | | |
|---|---|---|
| Compaction | 50-60% of Area left, | no compaction |
| | falls apart when touched | |

### Flowability

For examining flow properties, an Agway funnel flow test was done. In accordance with the procedure, a funnel [electropolished metal, thickness about 1.5 mm, conical configuration, one opening (orifice) having a 11.3 mm I.D. (inside diameter); 225 mm long; angle between its longitudinal axis and the wall is 10.5 degrees] was placed in a suitable ring stand with the orifice of the funnel located approximately 200 mm from a table top. 100 g of test material were weighed and poured into the above described funnel. Any loss of material from the funnel orifice was prevented by covering the orifice with a finger or plate. The material was permitted to flow from the funnel by removal of the plate and if needed slightly tapping the outside of funnel side wall with a finger or other rod like implement. The time required to empty the funnel was noted. The procedure was repeated at least three times, taking an average and recording it as the flow time. Analysis of the flow properties is based on the flow time recorded, the visual flow behavior, and the number of taps to the funnel side wall necessary to initiate or maintain flow. Flow or non-flow through the Agway funnel serves as a useful vehicle in measuring the relative flowability of a product and for predicting potential flow problems in e.g. emptying silos.

Example 1 shows that flowability of benzoic acid particles produced in prilling tower is high (even though non-spherical by-product had not yet been removed).

### Dusting

For determination of dust generated by the handling of benzoic acid particles, a commercially available dustmeter from Heubach® (Langelsheim, Germany) was used.

Example 1 shows that safety at work in the feed industry is significantly improved when using benzoic acid particles which are produced in prilling tower.

### Particle Size

Particle size as indicated in Table 1 was measured by Laser Diffraction; Malvern Mastersizer 2000, MIE volume distribution. The fundamental size distribution derived by this technique is volume based. This means that when the result indicates, for example, that 4% of the distribution is in the size category 0.600 mm-0.850 mm, this means that the total volume of all particles with diameters in this range represents 4% of the total volume of all particles in the distribution. D (v, 0.5), D (v, 0.1) and D (v, 0.9) are standard “percentile” readings from the analysis. D (v, 0.5) is the size in mm at which 50% of the sample is smaller and 50% is larger.

Example 1 shows that the benzoic acid particles produced in the prilling tower have a similar average particle size as commercially available benzoic acid flakes.

### Compaction Test

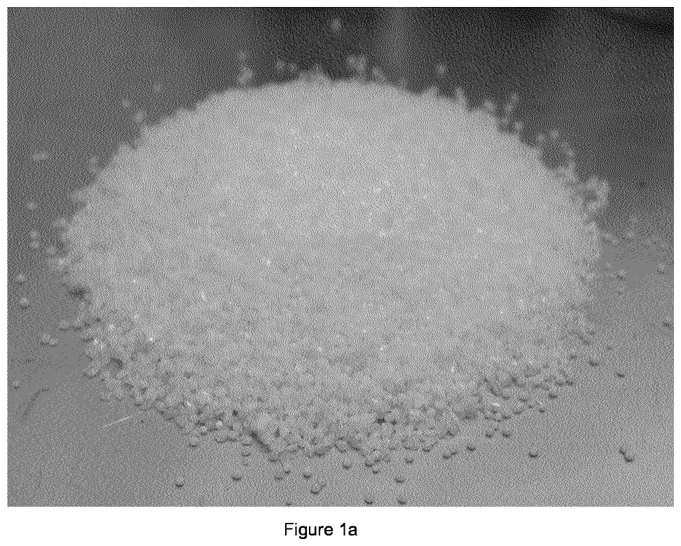

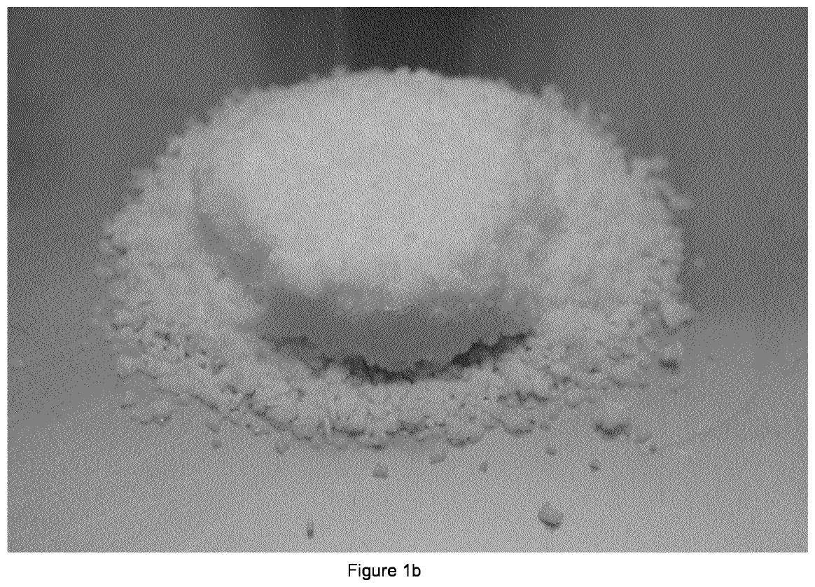

The result of the compaction test is shown in FIGS. 1a and 1b. For testing compaction, a metal tube (solid ring) with 5 cm bore diameter (≈20 cm²) was filled with 15 g of test material. For simulating weight, a metal cylinder with 5 cm diameter (weight is 1.25 kg corresponding to 0.625 N/cm²=6′250 N/m² simulating a weight of 625 kg on an area of 1 m²) was placed on the test material in the metal tube. This setting was then stored for 1 to 5 days at Room Temperature (RT≈25° C.)/ambient humidity (≈60%). The cylinder has then cautiously been removed and the product was assessed visually and with the aid of a spatula. Rating was then done with 2 scales: (1) Block size of compacted material after removal of weights expressed by percent of block circular area (surface) compared to tube diameter and (2) required force to separate the block.

Example 1 shows that the benzoic acid particles produced in the prilling tower are significantly less prone to compaction.

### Visual Inspection

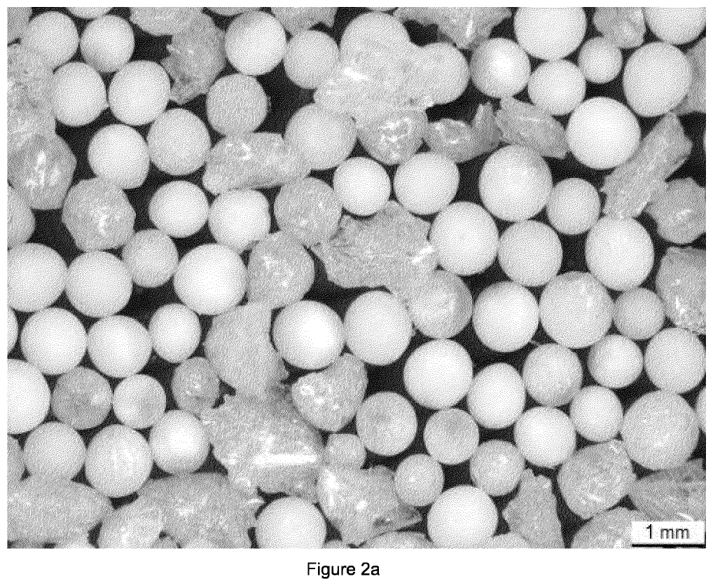

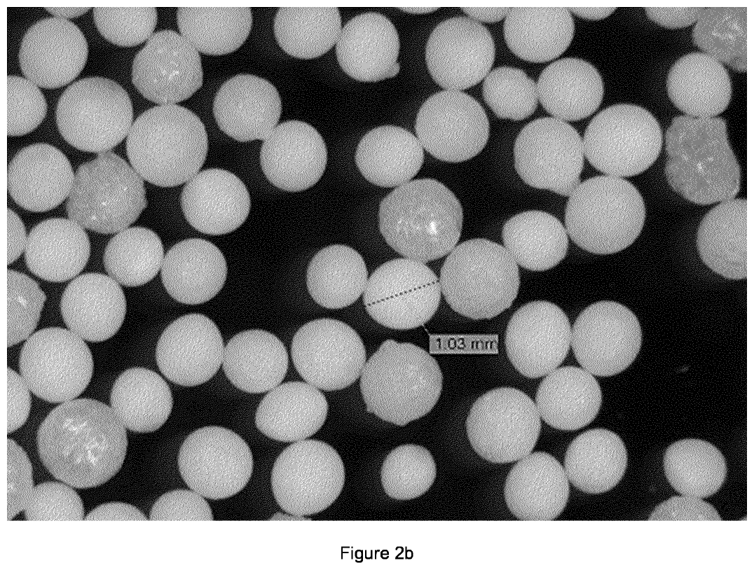

Microscopic inspection of the benzoic acid particles produced in the prilling tower as used in example 1 shows a mixture of spherical prills and uneven crystal-shaped particles (cf. FIG. 2a). Microscopic inspection of commercially available VevoVitall® benzoic acid flakes confirms the absence of spherical particles (cf. FIG. 2c).

### Example 2

In Example 2, non-spherical particles (by-product) contained in the benzoic acid particles which had been produced in the prilling tower, were removed. The thus obtained spherical benzoic acid prills are shown in FIG. 2b.

For removal of the non-spherical by-product, the benzoic acid particles which had been produced in the prilling tower were moved over an inclined surface. Spherical prills rolled like marbles and thus arrived faster than the non-spherical by-product at the bottom of the inclined surface. After this separation process, flowability, bulk density and tapped density was measured again. The result is shown in below TABLE 2:

TABLE 2

| | Benzoic acid | separated |
|---|---|---|
| | prills | by-product |
| | (“spherical | (“non-spherical |
| Sample | fraction”) | fraction”) |
| Flowability through Agway | 5 | 7 |
| funnel, orifice 11.3 mm | | |
| (sec/100 g) | | |
| Bulk density (kg/l) | 0.72 | 0.64 |
| Tapped density (kg/l) | 0.75 | 0.69 |

In case of separated by-product, it took 7 seconds to empty the Agway funnel. For the benzoic acid prills, it took only 5 second to the same Agway funnel under the same conditions. Thus, example 2 confirms that flowability of benzoic acid prills as herein described is high.

### Example 3

In Example 3, dissolution of various kinds of benzoic acid particles was tested. The samples whose dissolution rates were tested are further described in below TABLE 3:

TABLE 3

| sample | composition | representative picture |
|---|---|---|
| Benzoic acid prills | 100% benzoic | FIG. 2b |
| (“spherical fraction”) | acid | |
| separated by-product | 100% benzoic | cf. non-spherical particles |
| (“non-spherical fraction”) | acid | in FIG. 2a |
| benzoic acid flakes | 100% benzoic | FIG. 2c |
| (VevoVitall ®) | acid | |

Dissolution was measured in a USP-4 release tester (Sotax CE 7 smart). This device uses a flow cell. The following parameters were used in the dissolution test:
Test temperature: 37.0° C.
Pump delivery: 8 ml/min.
Cell: 12 mm
Buffer: HCl pH 2

Samples were taken inter alia after 5, 10, 15, 30 and 60 minutes and concentration of released benzoic acid was measured.

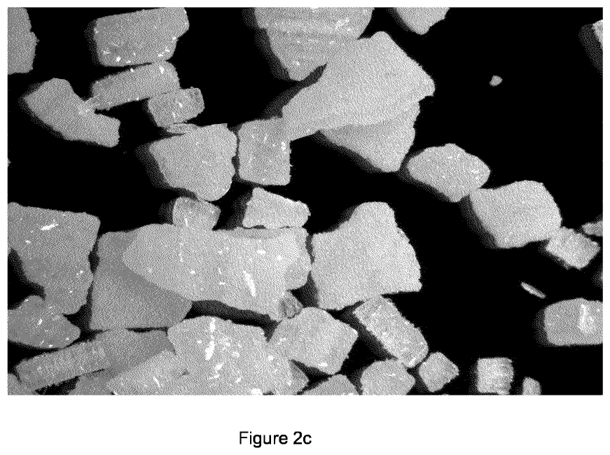

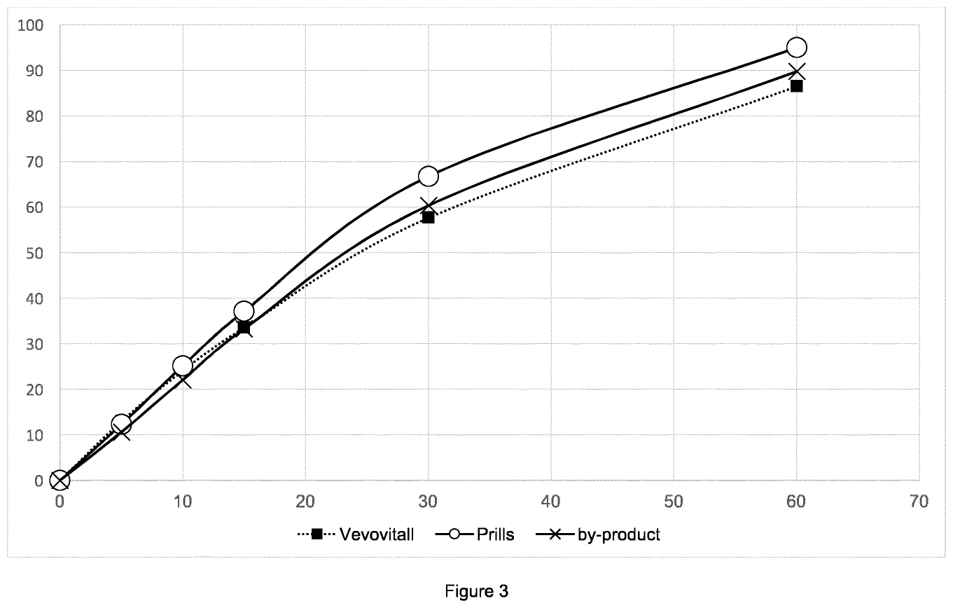

The result of the dissolution tests is shown in FIG. 3. Spherical benzoic acid prills dissolved faster than benzoic acid flakes whereas the separated, non-spherical by-product dissolved approximately as fast as the benzoic acid flakes (cf. FIG. 3). This is surprising as all samples are particles consisted of benzoic acid only.

### Example 4

In example 4, a feed additive was produced by mixing benzoic acid prills with a commercially available powder. The powder comprises a mixture of encapsulated essential oil. The volume of the mixture before and after tapping was the determined using a commercially available Jolting volumeter (J. Engelsmann A G, Ludwigshafen, Germany). An empty graduated 250 ml glass cylinder (accuracy 2 ml) was used.

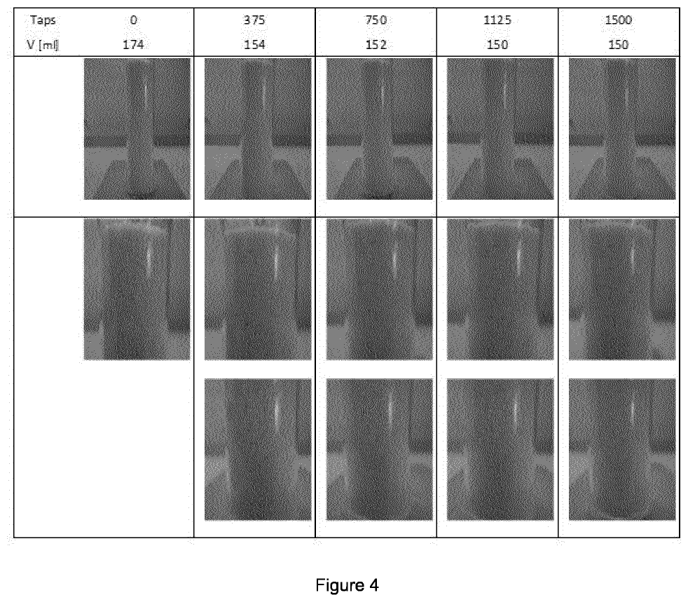

Before starting the test, a volume of 174 ml was tested. After 1125 taps the volume was reduced to 150 ml. The volume did not further decrease after a total of 1500 taps. FIG. 4 shows that the mixture in the glass volumeter did not show any signs of de-mixing, not even after 1500 taps.

Example 4 shows that benzoic acid prills are suitable for manufacturing feed additives, pre-mixes and feed.

1. Method of producing benzoic acid prills, said method comprising the steps:
(i) providing particles by spray cooling molten benzoic acid;
(ii) removing non-spherical particles from the particles provided in step (i).

2. Method according to claim 1, wherein step (i) is done by spraying molten benzoic acid into a gaseous cooling medium, and wherein said gaseous cooling medium is preferably air.

3. Method according to claim 1, wherein in step (ii), the difference between the sliding friction of non-spherical particles and the rolling friction of spherical particles is used for separating non-spherical particles from spherical particles.

4. Animal food comprising benzoic acid prills.

5. Animal food according to claim 4, wherein said animal food is a feed additive, a premix or feed.

6. Animal food according to claim 4, said animal food further comprising at least one essential oil, wherein said essential oil is preferably thymol and/or eugenol.

7. Process for the preparation of animal food, said process comprising the step of adding benzoic acid prills to a composition which can be fed to an animal.

8. Process according to claim 7, wherein said animal food is a feed additive, a premix or feed.

9. Use of benzoic acid prills for acidification of feed and/or urine.

10. Use according to claim 9, wherein the urine of an animal is acidified by feeding benzoic acid prills to the animal, and wherein said animal is preferably a domestic animal, and wherein said domestic animal is preferably a farm animal, and wherein said farm animal is preferably a chicken or a pig, and wherein said farm animal is most preferably a pig.

11. Animal food, process or use according to claim 4, wherein said benzoic acid prills comprise at least 95 weight-% benzoic acid, preferably at least 98 weight-% benzoic acid, more preferably at least 99 weight-% benzoic acid, even more preferably at least 99.5 weight-% benzoic acid and most preferably at least 99.9 weight-% benzoic acid, based on the total weight of the prills.

12. Animal food, process or use according to claim 11, wherein said benzoic acid prills are free of fat-soluble auxiliary compounds.

13. Animal food, process or use according to claim 11,
wherein said benzoic acid prills are spherical and/or wherein said benzoic acid prills have neither edges nor spikes.

14. Animal food, process or use according to claim 11, wherein said benzoic acid prills have an average particle size D (v,0.5) from 0.5 mm to 6 mm, more preferably from 0.5 mm to 5 mm and most preferably from 0.5 mm to 2 mm.

15. Animal food, process or use according to claim 11, wherein said benzoic acid prills are obtained.
